(19) United States
(12) Patent Application Publication
(10) Pub. No.: US 2025/0255966 A1
(43) Pub. Date: Aug. 14, 2025

(54) CORN PHYTOGLYCOGEN FORMULATIONS FOR STABILIZING PROTEINS

(71) Applicant: UNIVERSITY OF FLORIDA RESEARCH FOUNDATION, INCORPORATED, Gainesville, FL (US)

(72) Inventors: Susan Kim BOEHLEIN, Gainesville, FL (US); Gregory A. HUDALLA, Gainesville, FL (US); Renjie LIU, Gainesville, FL (US); Junha PARK, Gainesville, FL (US); Marcio F. RESENDE, Gainesville, FL (US)

(21) Appl. No.: 18/857,009

(22) PCT Filed: Apr. 17, 2023
(86) PCT No.: PCT/US2023/018868

§ 371 Date: Oct. 15, 2024

Related U.S. Application Data
(60) Provisional application No. 63/331,524, filed on Apr. 15, 2022.

(51) Int. Cl. A61K 47/26; A61K 9/00; A61K 45/06; A61K 47/10; A61K 47/32; A61K 47/34; A61K 47/38

(52) U.S. Cl. CPC A61K 47/26; A61K 9/0019; A61K 45/06; A61K 47/10; A61K 47/32; A61K 47/34; A61K 47/38

(57) ABSTRACT

Provided are formulations that utilize phytoglycogen as a stabilizing excipient. The phytoglycogen may be from maize, or more specifically maize harboring a mutation on the sugary1 (su1) gene. Also provided are methods of stabilizing a protein active ingredient by formulation the protein active ingredient with phytoglycogen.

### BACKGROUND

Aqueous formulations of proteins such as therapeutic proteins (e.g., antibodies) are susceptible to degradation through a number of different mechanisms and as a result of several types of stress conditions. In general, degradation of a therapeutic protein formulation occurs when the protein structure is altered slightly from its fully folded conformation (partial unfolding) exposing hydrophobic residues that interact with an adjacent protein molecule in solution forming an irreversible association. Certain stress conditions such as agitation, freeze/thaw and increased temperature can induce greater protein unfolding leading to accelerated aggregation of the protein and degradation of the protein formulation. Degradation of the protein formulation can be manifested by protein denaturation, the formation of visible particles, the formation of aggregates, the formation of subvisible particles, opalescence of the formulation, loss of biological activity, loss of percent monomer, loss of yield during production and purification, and the like. Exposure of the protein formulation to a liquid/air or liquid/solid interface, such as in agitation or freeze/thaw conditions, allows for a portion of the protein to unfold because of the lack of water at the interface to stabilize the folded structure through hydrogen bonding and hydrophobic effects. Other mechanisms leading to protein degradation include oxidation, hydrolysis, proteolysis, photodegradation, and microbial degradation. It would be desirable to provide a therapeutic protein formulation with improved stability to make the therapeutic proteins more resistant to the stress conditions encountered during their distribution and storage.

### SUMMARY

In certain embodiments, provided are formulations including a protein active ingredient and phytoglycogen as a stabilizing excipient. In a specific embodiment, the phytoglycogen is corn phytoglycogen. In other specific embodiments, the formulation may contain less than about 1 mg/mL of the protein active ingredient, or between about 1 μg/mL and about 1 mg/ml of protein active ingredient, or at least about 1 mg/ml of protein active ingredient, or at least about 5 mg/ml of protein active ingredient, or at least 100 mg/ml of protein active ingredient, or at least about 200 mg/ml of protein active ingredient, or at least about 300 mg/ml of protein active ingredient. Examples of the protein active ingredient include, but are not limited to, an antibody, an antibody-drug conjugate, an enzyme, a cytokine, a neurotoxin, a fusion protein, an immunogenic protein, a PEGylated protein, and an antibody fragment. In embodiments, the formulation contains at least about 1 to about 5000 ppm of the corn phytoglycogen, or at least about 1 to about 500 ppm of phytoglycogen, or at least about 10 to about 100 ppm of the phytoglycogen.

In certain embodiments, the formulations include an adjunct stabilizing excipient. The adjunct stabilizing excipient may include, but not be limited to, polypropylene glycol, adducts of polypropylene glycol, and random copolymers comprising propylene oxide units. In embodiments, the polypropylene glycol is a branched polymer, and the branched polymer can be formed by addition of propylene glycol units to a branched or multifunctional alcohol or a branched or multifunctional amine. The branched or multifunctional alcohol can be a sugar, glycerol, pentaerythritol, or triethanolamine. Moreover, the adjunct stabilizing excipient may include, but not be limited to, a hydrophobically modified cellulosic polymer can be selected from the group consisting of a methylcellulose, a hydroxypropyl methylcellulose, a hydroxypropyl cellulose, and a hydroxyethyl cellulose. Furthermore, the adjunct stabilizing excipient may be a polyvinyl alcohol, which can have a molecular weight between about 500 and about 500,000 Daltons, and/or which can have a hydrolysis percent between about 50% and about 100%. In yet other embodiments, the adjunct stabilizing excipient may be a polyoxazoline, which can be selected from the group consisting of poly(2-methyl-2-oxazoline), poly(2-ethyl-2-oxazoline) and poly(2-propyl-2-oxazoline). In embodiments, the polyoxazoline is poly(2-ethyl-2-oxazoline). In embodiments, the polyoxazoline has a weight-average molecular weight between about 1000 and about 500,000 Daltons, or a weight-average molecular weight between about 5000 and about 50,000 Daltons. In certain embodiments, the stabilizing excipient is polyvinylpyrrolidone, which can have a molecular weight between about 1000 and about 1,500,000 Daltons, or a molecular weight between about 5000 and about 200,000 Daltons, or a molecular weight between about 10,000 and about 100,000 Daltons.

In embodiments, the formulation can exclude conventional surfactants. In other embodiments, the formulation further comprises between about 1 and about 5000 ppm of a conventional surfactant, or it comprises between about 1 and about 100 ppm of the conventional surfactant, or it comprises between about 10 and about 5000 ppm of the conventional surfactant, or it comprises between about 100 and 2000 ppm of the conventional surfactant, or it comprises between about 100 and about 2000 ppm of the conventional surfactant. In other embodiments, the formulation further comprises an additional agent selected from the group consisting of preservatives, sugars, polysaccharides, arginine, proline, hyaluronidase, stabilizers, and buffers.

Also disclosed herein, in embodiments, are methods of improving stability in a formulation comprising a protein active ingredient by adding a stability-improving amount of a phytoglycogen to the formulation. In embodiments, the phytoglycogen reduces degradation of the formulation by at least 10%, as compared to a control formulation lacking the phytoglycogen, or the phytoglycogen reduces degradation of the therapeutic formulation by at least 30%, as compared to a control formulation lacking the phytoglycogen, or the phytoglycogen reduces degradation of the therapeutic formulation by at least 50%, as compared to a control formulation lacking the phytoglycogen, or the phytoglycogen reduces degradation of the therapeutic formulation by at least 70%, as compared to a control formulation lacking the phytoglycogen. Also disclosed herein, in embodiments, are methods of reducing adverse infusion-related effects in a patient, comprising administering to a patient in need thereof a therapeutic formulation comprising a protein active ingredient and a phytoglycogen, wherein infusing the therapeutic formulation into the patient results in fewer adverse infusion-related effects than infusing a control formulation into the patient, wherein the control formulation lacks the phytoglycogen. In embodiments, the adverse infusion-related effects are selected from the group consisting of adverse infusion reactions, adverse immunogenic responses, and decrease in half-life of a therapeutic protein in the therapeutic formulation.

### BRIEF DESCRIPTION OF THE DRAWINGS

FIG. 1. Properties of Phytoglycogen (PG). (a) TEM images of PG in different magnification. White globular sphere are PG. (b) Representative images of PG solubility test with 20 mg/mL PG in PBS. (c) Protein contaminants in soluble PG variants were separated by SDS PAGE. (d) Relative protein contaminant in PG variants measured via 660 assay. The upper right graph is magnified graph by a 20% y-axis scale

FIG. 2. Relative viability of various types of 20 mg/mL soluble PG at against NIH3T3 fibroblasts for 24 hours. Only soluble PG types are selected and evaluated. Dashed line indicates 80% viability which is general threshold for cytotoxicity test. (a) PG1, (b) PG2, (c) PG5, (d) PG8, (e) PG9, (f) PG11, (g) PG13, (h) PG15, (i) PG16.

FIG. 3. GFP preservation test with or without PG variants. PG concentration was fixed at 20 mg/mL, and GFP concentration was set at 4 μM, 8 μM, and 15 μM. Left images represent GFP fluorescence under UV light, C0, C10 means 0 and 10 cycles of lyophilization. Right graph indicates relative fluorescence comparison. Each of the images and absorbance measured before lyophilization (cycle 0), and after 10 cycles of lyophilization (cycle 10). (a) PG1, (b) PG2, (c) PG9, (d) PG13, (e) PG15, (f) PG16. The number above the graph indicates p-value.

FIG. 4. Lysozyme preservation test with or without PG variants. PG concentration was fixed at 20 mg/mL, and lysozyme concentration was varied between 0.25, 0.5, 1, and 2.5 mg/mL. Each of the absorbance measured before lyophilization (cycle 0), and after 10 times of lyophilization (cycle 10). (a) PG1, (b) PG2, (c) PG9, (d) PG13, (e) PG15, (f) PG16. The number above the graph indicates p-value.

FIG. 5. β-galactosidase (β-gal) and Horse radish peroxidase (HRP) preservation test with PG13. (a) β-gal concentration was varied between 300, 200, 100, and 50 μg/mL, while the PG13 concentration was fixed at 20 mg/mL. The relative activity was compared after cycle 4 against the initial activity (cycle 0). (b) HRP concentration was varied between 300, 200, 100, and 50 μg/mL, while the PG13 concentration was fixed at 20 mg/mL. The relative activity was compared after cycle 3 against the initial activity (cycle 0). The number above the graph indicates p-value.

FIG. 6. GFP preservation test with PG13 and common excipients. (a) GFP fluorescence under UV light. Cycle 0 and Cycle 10 fluorescence was compared. (b) Relative fluorescence was compared after cycle 10 against cycle 0. In the figure, G denotes glucose, (d) denotes dendrimer, (m) denotes monomer, (p) denotes polymer, (di) denotes disaccharide, Syn denotes synthetic polymer, and nG indicates non-glucose excipient.

FIG. 7. FT-IR measurements of 20 mg/mL PG2 with various concentration of lysozyme (2.5, 1, 0.5, and 0.25 mg/mL). (a) FT-IR graph of PG2 with lysozyme. Blue line and red line represents cycle 0 and cycle 10, respectively. Wavelength of 1700 to 1500 cm⁻¹ were cropped. (b) The conservation of structure after 10 cycles of lyophilization was evaluated by comparing the area under the curve of the FT-IR graphs for each lysozyme condition. The red cross indicates that the area under the curve could not be measured due to loss of shape.

FIG. 8. GFP preservation test according to various PG concentration. GFP concentration was fixed at 4 μM, PG concentration was varied between 15 to 0 mg/mL. Left images represent GFP fluorescence under UV light, C0, C10 means 0 and 10 cycles of lyophilization. Right graph indicates a relative fluorescence comparison after cycle 10 against cycle 0. (a) PG1, (b) PG2, (c) PG9, (d) PG13, (e) PG15, (f) PG16. The number above the graph indicates p-value.

FIG. 9. Lysozyme preservation test according to various PG concentration. Lysozyme concentration was fixed at 0.5 mg/mL, PG concentration was varied between 15 to 0 mg/mL. The relative activity was compared after cycle 10 against the initial activity (cycle 0). The number above the graph indicates p-value.

FIG. 10. β-galactosidase (β-gal) and Horse radish peroxidase (HRP) preservation test with different concentration of PG13. (a) PG13 concentration was varied between 20, 15, 10, 5, 2, and 1 mg/mL, while the β-gal concentration was fixed at 300 g/mL. The relative activity was compared after cycle 4 against the initial activity (cycle 0). (b) PG13 concentration was varied between 20, 15, 10, 5, 2, and 1 mg/mL, while the HRP concentration was fixed at 200 μg/mL. The relative activity was compared after cycle 3 against the initial activity (cycle 0). The number above the graph indicates p-value.

FIG. 11. Relative activity of HRP with PG1, 2, 9, 13, 15, 16, and PBS. Relative activity was assessed after 3 cycles of lyophilization against cycle 0. 20 mg/ml concentration of all PG variants and 200 μg/mL HRP concentration was used. The number above the graph indicates p-value.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the present disclosure, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Various references are cited throughout the disclosure. The teachings of the references cited herein are incorporated herein in their entirety.

For the purpose of this disclosure, the term “protein” refers to a sequence of amino acids (i.e., a polypeptide) typically having a molecular weight between about 1-3000 kiloDaltons (kDa). Polypeptides with molecular weight of about 1 kDa or higher are considered to be proteins for the purposes of the invention. In some embodiments, the molecular weight of the protein is between about 50-200 kDa; in other embodiments, the molecular weight of the protein is between about 20-1000 kDa or between about 20-2000 kDa. As would be understood by skilled artisans, a polypeptide of sufficient chain length can have a tertiary or quaternary structure, while shorter polypeptides can lack a tertiary or quaternary structure. A wide variety of biopolymers are included within the scope of the term “protein.” For example, the term “protein” can refer to therapeutic or non-therapeutic proteins, including antibodies, aptamers, fusion proteins, Fc fusion proteins, PEGylated proteins, synthetic polypeptides, protein fragments, lipoproteins, enzymes, immunogenic proteins (e.g., as used in vaccines), structural peptides, peptide drugs, and the like.

The term “protein active ingredient” as used herein refers to therapeutic proteins and non-therapeutic proteins.

Those proteins having therapeutic effects may be termed “therapeutic proteins”; formulations containing therapeutic proteins in therapeutically effective amounts may be termed “therapeutic formulations.” Typically, a therapeutic formulation comprises a therapeutically effective amount of a protein active ingredient and an excipient, with or without other optional components.

As used herein, the term “therapeutic” includes both treatments of existing disorders and preventions of disorders. A “treatment” includes any measure intended to cure, heal, alleviate, improve, remedy, or otherwise beneficially affect the disorder, including preventing or delaying the onset of symptoms and/or alleviating or ameliorating symptoms of the disorder. The term “treatment” includes a prophylactic or therapeutic vaccine or other preventive intervention.

Those patients in need of a treatment include both those who already have a specific disorder, and those for whom the prevention of a disorder is desirable. A disorder is any condition that alters the homeostatic wellbeing of a mammal, including acute or chronic diseases, or pathological conditions that predispose the mammal to an acute or chronic disease. Non-limiting examples of disorders include cancers, metabolic disorders (e.g., diabetes), allergic disorders (e.g., asthma), dermatological disorders, cardiovascular disorders, respiratory disorders, hematological disorders, musculoskeletal disorders, inflammatory or rheumatological disorders, autoimmune disorders, gastrointestinal disorders, urological disorders, sexual and reproductive disorders, neurological disorders, infectious diseases, and the like.

The term “mammal” for the purposes of treatment can refer to any animal classified as a mammal, including humans, domestic animals, pet animals, farm animals, sporting animals, working animals, and the like. A “treatment” can therefore include both veterinary and human treatments. For convenience, the mammal undergoing such “treatment” can be referred to as a “patient.” In certain embodiments, the patient can be of any age, including fetal animals in utero.

In embodiments, a treatment involves providing a therapeutically effective amount of a therapeutic formulation to a mammal in need thereof. A “therapeutically effective amount” is at least the minimum concentration of the therapeutic protein administered to the mammal in need thereof, to effect a treatment of an existing disorder or a prevention of an anticipated disorder (either such treatment or such prevention being a “therapeutic intervention”). Therapeutically effective amounts of various therapeutic proteins that may be included as active ingredients in the therapeutic formulation may be familiar in the art; or, for therapeutic proteins discovered or applied to therapeutic interventions hereinafter, the therapeutically effective amount can be determined by standard techniques carried out by those having ordinary skill in the art, using no more than routine experimentation.

Plant-derived glycogen, or “phytoglycogen” (PG) is a highly branched water-soluble polymer of glucosyl units. Plants utilize the plant polysaccharide in the same manner animals utilize glycogen: as energy for metabolic processes. A number of plants produce phytoglycogen, including corn, rice, sorghum, barley, and Arabidopsis. Phytoglycogen is a small particle (˜40-90 nm), monodisperse, has high stability in water, low viscosity and exceptional water retention. Phytoglycogen is also extracted from a common food source and biodegrades into simple sugars that are readily metabolized. While the list of recent publications and patents about PG is impressive, it is important to note that all the reported applications have evaluated one particular form of PG isolated from one mutant allele of sweet corn. Preliminary findings indicate that different varieties of sweet corn and different mutant alleles within genes of the starch biosynthesis pathway result in different water-soluble polysaccharides-all are termed phytoglycogen. See Liu et al., Molecules 2020, 25 (3), 637, incorporated by reference in its entirety. This Liu et al. reference teaches the extraction of PG from sweet corn varieties A632, A619, Wesu7, and Ia453. These varieties form PGs with different mammalian biocompatibility levels and particle sizes. PG of a certain variety can be selected based on its suitability for a given purpose, e.g., therapeutic or non-therapeutic applications, based on its biocompatibility (e.g. cytotoxicity). In corn, accumulation of phytoglycogen correlates with the deficiency of two types of debranching enzymes that hydrolyze PG α-1,6 linkages. A structural representation of phytoglycogen is shown below:

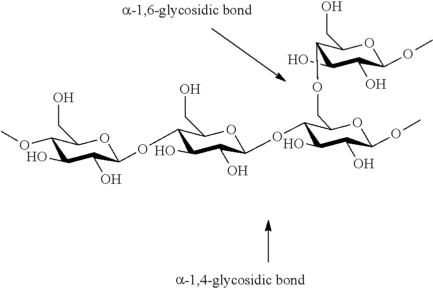

As non-limiting examples, therapeutic proteins can include mammalian proteins such as hormones and prohormones (e.g., insulin and proinsulin, synthetic insulin, insulin analogs, glucagon, calcitonin, thyroid hormones (T3 or T4 or thyroid-stimulating hormone), parathyroid hormone, gastrin, cholecystokinin, leptin, follicle-stimulating hormone, oxytocin, vasopressin, atrial natriuretic peptide, luteinizing hormone, growth hormone, growth hormone releasing factor, somatostatin, and the like); clotting and anti-clotting factors (e.g., tissue factor, von Willebrand's factor, Factor VIIIC, Factor VIII, Factor IX, protein C, plasminogen activators (urokinase, tissue-type plasminogen activators), thrombin); cytokines, chemokines, and inflammatory mediators (e.g., tumor necrosis factor inhibitors); interferons; colony-stimulating factors; interleukins (e.g., IL-1 through IL-10); growth factors (e.g., vascular endothelial growth factors, fibroblast growth factor, platelet-derived growth factor, transforming growth factor, neurotrophic growth factors, insulin-like growth factor, and the like); albumins; collagens and elastins; hematopoietic factors (e.g., erythropoietin, thrombopoietin, and the like); osteoinductive factors (e.g., bone morphogenetic protein); receptors (e.g., integrins, cadherins, and the like); surface membrane proteins; transport proteins; regulatory proteins; antigenic proteins (e.g., a viral component that acts as an antigen, as for example in a vaccine). A therapeutic protein can also be an immunogenic or other protein (including polypeptide) that is used as a vaccine, where a vaccine is a natural or synthetic preparation that induces acquired immunity to a disease. Therapeutic formulations used as vaccines include toxoid vaccines, protein-based or protein subunit-based vaccines, or conjugate vaccines. As an illustrative, non-limiting example, vaccines can contain a surface protein of a virus or a subunit thereof, as in the HPV virus, the Hepatitis B virus, and the influenza virus.

Therapeutic proteins used as vaccines may be derived from natural sources, for example, polypeptides or polypeptide fragments derived from microorganisms such as fungi (e.g., Aspergillus, Candida species), bacteria (e.g., Escherichia spp., Staphylococci spp., Streptococci spp.), protozoa such as sporozoa (e.g., Plasmodia), rhizopods (e.g., Entamoeba) and flagellates (Trypanosoma, Leishmania, Trichomonas, Giardia, etc.), and viruses, such as (+) RNA viruses, (−) RNA viruses, dsDNA viruses, RNA to DNA viruses, and DNA to RNA viruses. Examples of viruses from which vaccines are derived include without limitation Poxviruses (e.g., vaccinia), Picornaviruses (e.g., polio), Togaviruses (e.g., rubella), Flaviviruses (e.g., HCV); Coronaviruses, Rhabdoviruses (e.g., VSV); Paramyxovimses (e.g., RSV); Orthomyxovimses (e.g., influenza); Bunyaviruses; Arenaviruses, Reoviruses, retroviruses (e.g., HIV, HTLV); and Hepatitis B virus.

The term “therapeutic protein” includes, without limitation, the full complement of proteins that can be used as drugs, and include but are not limited, for example, fusion proteins such as etanercept, denileukin diftitox, alefacept, abatacept, rilonacept, romiplostim, corifollitropin-alpha, belatacept, aflibercept, ziv-aflibercept, eftrenonacog-alpha, albiglutide, efraloctocog-alpha, dulaglutide, and the like. The term “therapeutic protein” also includes antibodies.

The term “antibody” is used herein in its broadest sense, to include as non-limiting examples monoclonal antibodies (including, for example, full-length antibodies with an immunoglobulin Fc region), single-chain molecules, bi-specific and multi-specific antibodies, diabodies, antibody-drug conjugates, antibody compositions having polyepitopic specificity, and fragments of antibodies (including, for example, Fab, Fv, Fc, and F(ab′)2).

Antibodies can also be termed “immunoglobulins.” An antibody is understood to be directed against a specific protein or non-protein “antigen,” which is a biologically important material; the administration of a therapeutically effective amount of an antibody to a patient can complex with the antigen, thereby altering its biological properties so that the patient experiences a therapeutic effect.

In embodiments, the proteins can be PEGylated, meaning that they comprise polyethylene glycol (PEG) and/or polypropylene glycol (PPG) units. PEGylated proteins, or PEG-protein conjugates, have found utility in therapeutic applications due to their beneficial properties such as improved solubility, improved pharmacokinetics, improved pharmacodynamics, less immunogenicity, lower renal clearance, and improved stability. Non-limiting examples of PEGylated proteins are PEGylated interferons (PEG-IFN), PEGylated anti-VEGF, PEG protein conjugate drugs, Adagen, Pegaspargase, Pegfilgrastim, Pegloticase, Pegvisomant, PEGylated epoetin-β, and Certolizumab pegol.

PEGylated proteins can be synthesized by a variety of methods such as a reaction of protein with a PEG reagent having one or more reactive functional groups. The reactive functional groups on the PEG reagent can form a linkage with the protein at targeted protein sites such as lysine, histidine, cysteine, and the N-terminus. Typical PEGylation reagents have reactive functional groups such as aldehyde, maleimide, or succinimide groups that have specific reactivity with targeted amino acid residues on proteins. The PEGylation reagents can have a PEG chain length from about 1 to about 1000 PEG and/or PPG repeating units. Other methods of PEGylation include glyco-PEGylation, where the protein is first glycosylated and then the glycosylated residues are PEGylated in a second step. Certain PEGylation processes are assisted by enzymes like sialyltransferase and transglutaminase.

While the PEGylated proteins can offer therapeutic advantages over native, non-PEGylated proteins, these materials can have physical or chemical properties that make them difficult to purify, dissolve, filter, concentrate, and administer. The PEGylation of a protein can lead to a higher solution viscosity compared to the native protein, and this generally requires the formulation of PEGylated protein solutions at lower concentrations.

Those proteins used for non-therapeutic purposes (i.e., purposes not involving treatments), such as household, nutrition, commercial, research, and industrial applications, may be termed “non-therapeutic proteins.” Formulations containing non-therapeutic proteins may be termed “non-therapeutic formulations”. The non-therapeutic proteins can be derived from plant sources, animal sources, or produced from cell cultures; they also can be enzymes or structural proteins. The non-therapeutic proteins can be used in household, nutrition, commercial, and industrial applications such as catalysts, human and animal nutrition, processing aids, cleaners, and waste treatment.

An important category of non-therapeutic biopolymers includes enzymes. Enzymes have a number of non-therapeutic applications, for example, as catalysts, human and animal nutritional ingredients, processing aids, cleaners, and waste treatment agents. Enzyme catalysts are used to accelerate a variety of chemical reactions. Examples of enzyme catalysts for non-therapeutic uses include catalases, oxidoreductases, transferases, hydrolases, lyases, isomerases, and ligases. Human and animal nutritional uses of enzymes include nutraceuticals, nutritive sources of protein, chelation or controlled delivery of micronutrients, digestion aids, and supplements; these can be derived from amylase, protease, trypsin, lactase, and the like. Enzymatic processing aids are used to improve the production of food and beverage products in operations like baking, brewing, fermenting, juice processing, and winemaking. Examples of these food and beverage processing aids include amylases, cellulases, pectinases, glucanases, lipases, and lactases. Enzymes can also be used in the production of biofuels. Ethanol for biofuels, for example, can be aided by the enzymatic degradation of biomass feedstocks such as cellulosic and lignocellulosic materials. The treatment of such feedstock materials with cellulases and ligninases transforms the biomass into a substrate that can be fermented into fuels. In other commercial applications, enzymes are used as detergents, cleaners, and stain lifting aids for laundry, dishwashing, surface cleaning, and equipment cleaning applications. Typical enzymes for this purpose include proteases, cellulases, amylases, and lipases. In addition, non-therapeutic enzymes are used in a variety of commercial and industrial processes such as textile softening with cellulases, leather processing, waste treatment, contaminated sediment treatment, water treatment, pulp bleaching, and pulp softening and debonding. Typical enzymes for these purposes are amylases, xylanases, cellulases, and ligninases.

Other examples of non-therapeutic biopolymers include fibrous or structural proteins such as keratins, collagen, gelatin, elastin, fibroin, actin, tubulin, or the hydrolyzed, degraded, or derivatized forms thereof. These materials are used in the preparation and formulation of food ingredients such as gelatin, ice cream, yogurt, and confections; they are also added to foods as thickeners, rheology modifiers, mouthfeel improvers, and as a source of nutritional protein. In the cosmetics and personal care industry, collagen, elastin, keratin, and hydrolyzed keratin are widely used as ingredients in skin care and hair care formulations. Still other examples of non-therapeutic biopolymers are whey proteins such as beta-lactoglobulin, alpha-lactalbumin, and serum albumin. These whey proteins are produced in mass scale as a byproduct from dairy operations and have been used for a variety of non-therapeutic applications.

As used herein, the term “conventional surfactant” refers to an organic surface-active agent capable of lowering the surface tension between two liquids, or lowering the interfacial tension between a liquid and a solid. A conventional surfactant is typically amphiphilic, and can include a hydrophilic “head” and one or two hydrophobic “tails.” The charged character of the head group allows categorization of the conventional surfactant: a surfactant with a positively-charged head is termed cationic; a surfactant with a negatively-charged head is termed anionic; a surfactant with no charged groups on its head is termed non-ionic; and a surfactant having a head with two oppositely charged groups is termed zwitterionic. The tail of the conventional surfactant can comprise a branched, linear, or aromatic hydrocarbon chain, or it can comprise a fluorocarbon chain (for fluorosurfactants), or a siloxane chain (for siloxane surfactants). The hydrophilic properties of a conventional surfactant can be increased by including ethoxylated sequences (e.g. polyethylene oxide), while the lipophilic properties of the conventional surfactant can be increased by including polypropylene oxide sequences.

In embodiments, the conventional surfactant can be a polysorbate, i.e., an emulsifier derived from an ethoxylated sorbitan ester of a fatty acid. For example, Polysorbate 20 (polyoxyethylene (20) sorbitan monolaurate) and Polysorbate 80 (polyoxyethylene (20) sorbitan monooleate) are commonly used as conventional surfactants for protein formulations. In other embodiments, the conventional surfactant can be an ethoxylated fatty alcohol, a diblock copolymer of ethylene oxide (EO) and propylene oxide (PO), or a triblock copolymer of EO and PO.

The term “adjunct stabilizing excipient” as used herein refers to one or more stabilizing excipients that are not phytoglycogen.

Other definitions relating to invention are provided infra.

### 2. General

The present disclosure relates to aqueous formulations of therapeutic or non-therapeutic proteins with stabilizing excipients. As used herein, the term “stabilizing excipient” refers to an excipient that reduces the degradation of a therapeutic protein in response to a stress condition. Phytoglycogen, and in particular, corn phytoglycogen, has newly been discovered as useful as a stabilizing excipient. A stress condition can be any condition that alters the protein structure, for example, by causing greater protein unfolding, leading to accelerated aggregation and degradation of the protein formulation. Stress conditions can include, without limitation, agitation, filtration, freeze/thaw conditions, lyophilization, exposure to storage temperatures above 5° C., or exposure to a liquid/air or liquid/solid interface. Other mechanisms involved in stress conditions include oxidation, hydrolysis, proteolysis, deamidation, disulfide scrambling, photodegradation, and microbial degradation.

It is well known to those skilled in the art of polymer science and engineering that proteins in solution tend to form entanglements, which can limit the translational mobility of the entangled chains and interfere with the protein's therapeutic or nontherapeutic efficacy. In embodiments, stabilizing excipient compounds as disclosed herein can suppress protein clustering due to specific interactions between the excipient compound and the therapeutic protein in solution.

In embodiments, the approaches disclosed herein can yield a liquid formulation having improved stability when compared to a traditional protein solution. A stable formulation is one in which the protein contained therein substantially retains its physical and chemical stability and its therapeutic or nontherapeutic efficacy upon storage under storage conditions, whether cold storage conditions, room temperature conditions, or elevated temperature storage conditions. Advantageously, a stable formulation can also offer protection against aggregation or precipitation of the proteins dissolved therein. For example, the cold storage conditions can entail storage in a refrigerator or freezer. In some examples, cold storage conditions can entail conventional refrigerator or freezer storage at a temperature of 10° C. or less. In additional examples, the cold storage conditions entail storage at a temperature from about 2° to about 10° C. In other examples, the cold storage conditions entail storage at a temperature of about 4° C. In additional examples, the cold storage conditions entail storage at freezing temperature such as about 0° C. or lower. In another example, cold storage conditions entail storage at a temperature of about −30° C. to about 0° C. The room temperature storage conditions can entail storage at ambient temperatures, for example, from about 10° C. to about 30° C. Elevated temperature stability, for example, at temperatures from about 30° C. to about 50° C., can be used as part of an accelerated aging study to predict the long term storage at typical ambient (10-30° C.) conditions.

In embodiments, formulations include one or more adjunct stabilizing excipients in addition to phytoglycogen. In certain embodiments, adjunct stabilizing excipients may include one or more of propylene glycol, polypropylene glycol homopolymers, adducts of polypropylene glycol, or random copolymers comprising propylene oxide units. In other embodiments, the adjunct stabilizing excipients can comprise a hydrophobically modified cellulose, which can be a methylcellulose, a hydroxypropyl methylcellulose, a hydroxypropyl cellulose, or a hydroxyethyl cellulose, and is not a sodium carboxymethyl cellulose. In other embodiments, the adjunct stabilizing excipient is polyvinyl alcohol. In other embodiments, the adjunct stabilizing excipient is a polyoxazoline, such as poly(2-ethyl-2-oxazoline). In other embodiments, the adjunct stabilizing excipient is polyvinyl pyrrolidone. Combinations of the foregoing adjunct stabilizing excipients may be selected for certain formulations.

The stabilizing excipient can be added alone, or in combination with conventional surfactants such as nonionic surfactants such as Polysorbate 80, Polysorbate 20 and the like. When a stabilizing excipient is combined with a conventional surfactant excipient, a lesser amount of conventional excipient may be required, for example. 0-100 ppm of the conventional surfactant, or 100-2000 ppm of the conventional surfactant. In other embodiments, the therapeutic protein formulation contains the stabilizing excipient and an amount of 10-5000 ppm of a conventional surfactant. In embodiments, the stabilizing excipient is added to the formulation in amounts ranging from 10-5000 ppm. In embodiments, the stabilizing excipient is added to the formulation in amounts ranging from 100-1000 ppm. Reducing the amounts of conventional surfactant in a therapeutic formulation can offer certain advantages such as improved formulation stability, improved excipient stability, and reduced foaming tendency. In embodiments, solutions of therapeutic proteins containing the stabilizing excipients of the invention can have a lower foaming tendency compared with solutions of the same therapeutic proteins without the stabilizing excipients.

Advantageously, the stabilizing excipients can be selected so that they do not form micelles in aqueous solution and they can pass through an ultrafiltration membrane. Advantageously, the stabilizing excipients can be selected so that they do not increase the foaming tendency of the formulation. Advantageously, the stabilizing excipients can be selected so that they do not include conventional amphiphilic surfactant structures. In embodiments, the stabilizing excipients can be selected so that they are not structured as having a hydrophilic head and a hydrophobic tail. In other embodiments, the stabilizing excipients can be selected so that they do not comprise block copolymers, for example, so that block copolymer arrangements such as the (propylene oxide-co-ethylene oxide) copolymer configurations of PO/EO/PO, EO/PO or EO/PO/EO are excluded. In embodiments, stabilizing excipients can be selected that are free of ethylene oxide (EO) groups, residual ethylene oxide monomer, and/or dioxane byproducts. In embodiments, the stabilizing excipients are selected so that they contain no ester linkages. In embodiments, the stabilizing excipients are purified to minimize the presence of endotoxins or heavy metals. In embodiments, the stabilizing excipients are USP grade materials. Stabilizing excipient compounds as disclosed herein can be natural or synthetic, and, in certain embodiments they may be substances that the U.S. FDA generally recognizes as safe (GRAS), or that are well established and commonly used in registered drug products such as are usually included in pharmacopoeias, or that are included in a registry or database such as the FDA's Inactive Ingredient Database (https://www.accessdata.fda.gov/scripts/cder/iig/).

### 3. Therapeutic Formulations

In one aspect, the formulations and methods disclosed herein provide stable liquid formulations, comprising a therapeutic protein in a therapeutically effective amount and phytoglycogen. In embodiments, the formulation can improve the stability while providing an acceptable concentration of active ingredients and an acceptable stability. In embodiments, the formulation provides an improvement in stability when compared to a control formulation; for the purposes of this disclosure, a control formulation is a formulation containing the protein active ingredient that is identical on a dry weight basis in every way to the therapeutic formulation except that it lacks the excipient compound. In embodiments, improved stability of the protein containing formulation is indicated by a lower percentage of soluble aggregates, a lower percentage of fragments, a decrease in the number of particulates, a decrease in the number of subvisible particles, a decrease in the hydrodynamic particle size, or the suppression of gel formation, as compared to a control formulation after a stress condition. In embodiments, the stress conditions can include freeze/thaw cycles, exposure to storage conditions for >1 month at freezing temperatures (below 0° C.), exposure to storage conditions for >1 month at refrigerated temperatures (between 0° C. and 15° C.), exposure to storage conditions for >1 month at ambient temperatures (between 15° C. and 30° C.), exposure to storage conditions for >1 week at elevated temperatures (between 30° C. and 100° C.), exposure to agitation stress, exposure to air/water interfaces, contact with plastic, glass, or metal surfaces, filtration, column chromatography separation, viral inactivation, exposure to pH conditions between pH 2 and pH 5, exposure to pH conditions between pH 8 and pH 12, exposure to proteolytic enzymes, exposure to lipase enzymes, or exposure to microbiological contamination.

It is understood that the stability of a liquid protein formulation can be affected by a variety of factors, including, but not limited to: the nature of the protein itself (e.g., enzyme, antibody, receptor, fusion protein, etc.); its size, three-dimensional structure, chemical composition, and molecular weight; its concentration in the formulation; the components of the formulation besides the protein; the formulation pH range; the storage conditions for the formulation; and the method of administering the formulation to the patient. Therapeutic proteins most suitable for use with the excipient compounds described herein are preferably essentially pure, i.e., free from contaminating proteins. In embodiments, an “essentially pure” therapeutic protein is a protein composition comprising at least 90% by weight of the therapeutic protein, or preferably at least 95% by weight, or more preferably, at least 99% by weight, all based on the total weight of therapeutic proteins and contaminating proteins in the composition. For the purposes of clarity, a protein added as an excipient is not intended to be included in this definition. The therapeutic formulations described herein are intended for use as pharmaceutical-grade formulations, i.e., formulations intended for use in treating a mammal, in such a form that the desired therapeutic efficacy of the protein active ingredient can be achieved, and without containing components that are toxic to the mammal to whom the formulation is to be administered.

In embodiments, the therapeutic formulation contains at least 1 μg/mL of protein active ingredient. In embodiments, the therapeutic formulation contains between about 1 μg/mL and about 10 mg/mL of protein active ingredient. In embodiments, the therapeutic formulation contains at least 1 mg/ml of protein active ingredient. In other embodiments, the therapeutic formulation contains at least 5 mg/ml of protein active ingredient. In other embodiments, the therapeutic formulation contains at least 100 μg/mL of protein active ingredient. In other embodiments, the therapeutic formulation contains at least 200 μg/mL of protein active ingredient. In yet other embodiments, the therapeutic formulation solution contains at least 300 μg/mL of protein active ingredient. Generally, the excipient compounds disclosed herein are added to the therapeutic formulation in an amount between about 1 to about 5000 ppm. In embodiments, the excipient compound can be added in an amount of about 1 to about 500 ppm. In embodiments, the excipient compound can be added in an amount of about 10 to about 100 ppm.

In embodiments, the excipient compounds disclosed herein are added to the therapeutic formulation in a stability-improving amount. In embodiments, a stability-improving amount is the amount of an excipient compound that reduces the degradation of the formulation by at least 10% when compared to a control formulation; for the purposes of this disclosure, a control formulation is a formulation containing the protein active ingredient that is identical on a dry weight basis in every way to the therapeutic formulation except that it lacks the excipient compound. In embodiments, the stability-improving amount is the amount of an excipient compound that reduces the degradation of the formulation by at least 30% when compared to the control formulation. In embodiments, the stability-improving amount is the amount of an excipient compound that reduces the degradation of the formulation by at least 50% when compared to the control formulation. In embodiments, the stability-improving amount is the amount of an excipient compound that reduces the degradation of the formulation by at least 70% when compared to the control formulation. In embodiments, the stability-improving amount is the amount of an excipient compound that reduces the degradation of the formulation by at least 90% when compared to the control formulation.

Therapeutic formulations in accordance with this disclosure have certain advantageous properties. In embodiments, the therapeutic formulations are resistant to shear degradation, phase separation, clouding out, precipitation, and denaturing. In embodiments, the therapeutic formulations are processed, purified, stored, syringed, dosed, filtered, and centrifuged more effectively, compared with a control formulation. In embodiments, the therapeutic formulations can result in fewer adverse infusion-related effects, for example, adverse infusion reactions, adverse immunogenic responses, decrease in half-life of a therapeutic protein in the therapeutic formulation, and the like. In embodiments, when the therapeutic formulations are administered to patients, they can experience fewer infusion reactions than would be experienced with a similar formulation lacking the stabilizing excipient. In embodiments, when the therapeutic formulations are administered to patients, they can experience fewer or less intense immunogenic responses than would be experienced with a similar formulation lacking the stabilizing excipient. In embodiments, when the therapeutic formulations are administered to patients, they can experience less decrease in the half-life of the therapeutic protein in the body, as compared to a similar formulation lacking the stabilizing excipient.

In embodiments, the therapeutic formulation is stored at ambient temperatures, or for extended time at refrigerator conditions without appreciable loss of potency for the therapeutic protein. In embodiments, the therapeutic formulation is dried down for storage until it is needed; then it is reconstituted with an appropriate solvent, e.g., water. Advantageously, the formulations prepared as described herein can be stable over a prolonged period of time, from months to years. When exceptionally long periods of storage are desired, the formulations can be preserved in a freezer (and later reactivated) without fear of protein denaturation. In embodiments, formulations can be prepared for long-term storage that do not require refrigeration. In embodiments, the stabilizing excipient can be used to improve solubility or stability of protein therapeutics that have limited water solubility, such as antibody-drug conjugates.

In embodiments, the stabilizing excipient provides a substitute for some or all of the conventional surfactants that are employed in protein formulations, as described previously. As described previously, the stabilizing excipient can be added to a protein formulation alone or in combination with one or more other excipients, either to replace the conventional surfactant in the formulation entirely, or to reduce the amount of the conventional surfactant that is used. In embodiments, the stabilizing excipient is not an ethoxylated compound, and does not contain residual amounts of 1,4-dioxane.

Methods for preparing therapeutic formulations may be familiar to skilled artisans. The therapeutic formulations of the present invention can be prepared, for example, by adding phytoglycogen to the formulation before or after the therapeutic protein is added to the solution. The therapeutic formulation can, for example, be produced by combining the therapeutic protein and the excipient at a first (lower) concentration and then processed by filtration or centrifugation to produce a second (higher) concentration of the therapeutic protein. Therapeutic formulations can be made with one or more of the excipient compounds with chaotropes, kosmotropes, hydrotropes, and salts. Therapeutic formulations can be made with one or more of the excipient compounds using techniques such as encapsulation, dispersion, liposome, vesicle formation, and the like. Methods for preparing therapeutic formulations comprising the stabilizing excipient compounds disclosed herein can include combinations of the excipient compounds. Other additives may be introduced into the therapeutic formulations during their manufacture, including preservatives, conventional surfactants, sugars, sucrose, trehalose, polysaccharides, arginine, proline, hyaluronidase, stabilizers, buffers, and the like. As used herein, a pharmaceutically acceptable stabilizing excipient compound is one that is non-toxic and suitable for animal and/or human administration.

The formulations of the present invention may be for parenteral administration, such as intravenous, intra-arterial, subcutaneous, intradermal, or intramuscular administration (e.g., by injection or by infusion). In some embodiments, the formulation is administered subcutaneously. The formulations can also be delivered transdermally, such as by topically applying the composition to skin (e.g., spreading the composition on skin or loading the composition onto a dermal patch and attaching the dermal patch to the skin).

The formulations of the present disclosure can be administered by infusion or by injection using any suitable device. For example, a formulation of the present invention may be placed into a syringe (e.g., a pre-filled syringe), a pen injection device, an auto-injector device, or a pump device. In some embodiments, the injection device is a multi-dose injector pump device or a multi-dose auto-injector device. The formulation is presented in the device in such a fashion that the formulation is readily able to flow out of the needle upon actuation of an injection device, such as an auto-injector, in order to deliver the peptide drugs. Suitable pen/auto injector devices include, but are not limited to, those pen/auto injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals and the like.

In some embodiments, the formulations of the present invention are provided ready for administration in a vial, a cartridge, or a pre-filled syringe.

Kits are also contemplated as being used in certain aspects of the present invention. For instance, a formulation of the present invention can be included within a kit. A kit can include a container. In one aspect, for instance, the formulation can be comprised within a container that is ready to administer to a subject without having to reconstitute or dilute the formulation. That is, the formulation to be administered can be stored in the container and be readily used as needed. The container can be a device. The device can be a syringe (e.g. pre-filled syringe), a pen injection device, an auto-injector device, a device that can pump or administer the formulation (e.g., automatic or non-automatic external pumps, implantable pumps, etc.) or a perfusion bag. Suitable pen/auto-injector devices include, but are not limited to, those pen/auto-injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals and the like.

### EXAMPLES

### Example 1: Methods and Materials

### Solubility Test

Solubility of different phytoglycogens (PGs) from different lines was tested by dissolving each in PBS at a weight/volume ratio of 20 mg/mL, sonicating for 30 to 40 minutes, and filtering through a 0.22 μm syringe filter (MilliporeSigma, USA). Samples with visible sedimented precipitate were excluded as insoluble PG, while samples with a clear solution or stable cloudy suspension were qualified as soluble PG. Samples were obtained from maize having different genotypes where the gene was mutated to knock down expression of the targeted gene. Test PG samples and the corresponding gene mutations are provided below (du1=dull1, isa2=isoamylase2, su1=sugary1, su2=sugary2; zpu1=pullanase-type starch debranching enzyme; the hyphenated information relates to the mutated allele)

### Mutations

PG14 du1-M3/isa2-339
PG6 du1-Ref/su1-P
PG4 du1-M3/su1-P
PG2 su2-Ref/su1-P
PG13 su1-st/isa2-339
PG12 su1-st/su2-2279
PG11 su1-st/zpu1-204
PG9 su1-Bn/isa2-339
PG3 su1-Ref/isa2-339
PG10 su1-P/su2-1981
PG8 su1-P/isa2-339
PG1 su1-Ref
PG7 su1-st
PG5 su1-Bn
PG15 su1
PG16 su1

Further support and characterization for the alleles is provided in Plant Physiol. 2012 February; 158 (2): 679-692 (du1-Ref and isa2-339); Plant Physiol. 2001 March; 125 (3): 1406-18 (su1-st); doi.org/10.2135/cropsci2015.11.0723 (su1-ref), Plant Physiol. 2010 July; 153 (3): 956-69 (su1-P), Plant Mol Biol. 2004 April; 54 (6): 865-79 (su2-2279 and su1-Ref), Plant Physiology, Volume 158, Issue 2, February 2012, Pages 679-692 (du1-M3)

### Toxicity Test

Toxicity was tested with NIH 3T3 fibroblast cells. Cells were cultured at 37° C. and 5% CO₂ in Dulbecco's Modified Eagle Medium (DMEM) (Gibco, USA) supplemented with 10% (v/v) fetal bovine serum (Biochrom, Germany), 1% penicillin-streptomycin. Cells were seeded in a 96-well plate (2×10⁴ cells/well) in 100 μL media, and treated with different types (PG1 to PG16) and concentrations (0, 1, 2, 5, 10, 20 mg/ml) of 100 μL PG. At t=24 hours, 20 μL of CellTiter-Blue cell viability reagent (Promega, USA) was added, and fluorescence at ex/em=560/590 nm was measured by a SpectraMax M3 plate reader at 1, 2, 3, and 4 h.

### Protein Contaminant Test

Protein contaminants in PG preparations were qualitatively assessed using SDS-PAGE and quantitatively measured using 660 nm assay (Pierce). SDS-PAGE buffer was prepared by adding 50 μL of 2-Mercaptoethanol into 950 μL of 2× Laemmli sample buffer (Bio-rad, USA). PG was dissolved in PBS and filtered as above. PG solutions were incubated at 95° C. for 5 minutes and immediately cooled down in ice to prevent reforming of secondary structure. Mini-PROTEAN® TGX™ Precast Gels (Bio-rad, USA) was set up, and lanes were loaded with 10 μL of PG1, 2, 9, 13, 15, 16 and Fisher BioReagents™ Routine DNA Ladder solution (Fisher scientific, USA). Samples were electrophoresed with a 150 kV field for 45 minutes. The gel was stained with the Coomassie Blue staining solution for 2 hours and washed 3 times with destaining buffer for 1 hour.

The 660 nm assay was conducted according to the manufacturer's instructions. A standard curve was established within the working range of the assay in wells of a clear-bottom 96-well plate. 10 μL of each filtered PG was added to wells of the same 96-well plate (Fisher scientific, USA). Subsequently, 150 μL of reagent was added to each standard and PG sample well and mixed using a plate shaker. After 5 minutes of incubation at room temperature, the absorbance of samples was measured at 660 nm using a SpectraMax M3 plate reader (Molecular devices, USA).

### Preservation Test (Fixed PG Concentration)

Protein preservation activity of PG was measured with lysozyme, green fluorescence protein (GFP), horse radish peroxidase (HRP), and beta-galactosidase (β-Gal) using a multi-cycle lyophilization assay.

To measure relative fluorescence of GFP after 10 cycles of lyophilization, PG concentration was fixed at 20 mg/mL while protein concentration was varied. 50 μL of 15, 8, 4 μM GFP was mixed either 50 μL of 20 mg/ml of PG or PBS as a control, at a 1:1 ratio. For one “cycle”, the mixture was frozen at −80° C., lyophilized, and resuspended in 100 μL of deionized water (DIW). This process was repeated for up to cycles. After 0 (control), 5, and 10 cycles, GFP fluorescence was measured at ex/em=485/510 nm using a SpectraMax M3 plate reader. Relative fluorescence was calculated by taking the ratio of the fluorescence at cycle 5 and cycle 10 versus the fluorescence at cycle 0.

To measure relative enzyme activity after lyophilization, PG concentration was fixed at 20 mg/mL while protein concentration was varied (2.5, 1, 0.5, and 0.25 mg/mL of lysozyme; 300, 200, 100, and 50 g/mL HRP; 300, 200, 100, and 50 μg/mL β-Gal). Lysozyme activity was measured after 10 cycles of lyophilization by EnzChek™ Lysozyme Assay Kit (Invitrogen, USA). 100 μL of DIW was added to rehydrate samples. 50 μL of each sample was added to wells of a black 96-well plate (Corning, USA). 50 μL of DQ lysozyme substrate was added to each well, and fluorescence at 494/518 nm was measured using the plate reader.

HRP activity was measured using the 3,3′,5,5′-Tetramethylbenzidine (TMB) (BD OptEIA, USA) assay. 100 μL of DIW was added to rehydrate samples. Each sample was then diluted 100-fold and 4 μL of each sample was transferred into wells of a clear-bottom 96-well plate. Then 50 μL of TMB solution was added to each well and maintained at room temperature for 1 min. 50 μL of 1M phosphoric acid (BD OptEIA, USA), was then added to quench the reaction. The absorbance of each solution was then read at 450 nm using a SpectraMax M3 plate reader.

β-Gal activity was measured using the O-nitrophenyl-β-D-galactoside (ONPG) (Thermo scientific) assay. 100 μL of DIW was added to rehydrate samples. 50 μL of each sample was transferred into wells of a clear-bottom 96-well plate. 7.5 μL of ONPG was then added to each sample. The absorbance of each sample was read at 405 nm for 30 mins using a SpectraMax M3 plate reader.

### Preservation Test (Fixed Protein Concentration)

To determine the effective PG concentration threshold, PG concentration was varied to 15, 10, 5, 2, and 1 mg/mL, while protein concentration was fixed. Each protein concentration was selected from the “Fixed PG” experiments above based on the group with the highest activity after 10 cycles of lyophilization (0.5 mg/ml lysozyme; 4 UM GFP; 200 μg/mL HRP; 300 μg/mL β-Gal). Each protein was mixed with 15, 10, 5, 2, and 1 mg/ml of PG. Samples were lyophilized, activity was measured, and relative activity was calculated using the same methods as above.

Comparison with Common Excipients

In order to investigate the stabilizing ability of PG compared to other commonly used excipients, seven different excipients were selected: PG13, glucose, dextran 40 kDa, sucrose, trehalose, polyethylene glycol (PEG) 1.5 kDa, PEG 6 kDa, and bovine serum albumin (BSA). As a model protein, 4 μM GFP was used. Each excipient was prepared at a concentration of 20 mg/mL as described above. Each excipient was mixed with GFP at a 1:1 (v/v) ratio. PBS without excipient was used as a control group. Samples were lyophilized, activity was measured, and relative activity was calculated using the same methods as above.

### Structural Analysis (FT-IR)

The secondary structure of each protein was analyzed using Fourier transform infrared (FT-IR) spectroscopy. After 0, 5, and 10 cycles, samples were rehydrated with 100 μL of DIW. 10 μL of each sample was transferred onto the FT-IR stage, and the absorbance spectrum was measured over the range of 400-4000 cm⁻¹. Absorbance at 1650 cm⁻¹ represents amide 1 band, and absorbance at 1550 cm⁻¹ corresponds to the amide 2 band. The ratio of the area under the amide 1 band versus the area under the amide 2 band was used to compare the secondary structure of the samples after cycle 10 versus cycle 0 in the presence and absence of PG.

### Example 2: Results

### Solubility Test

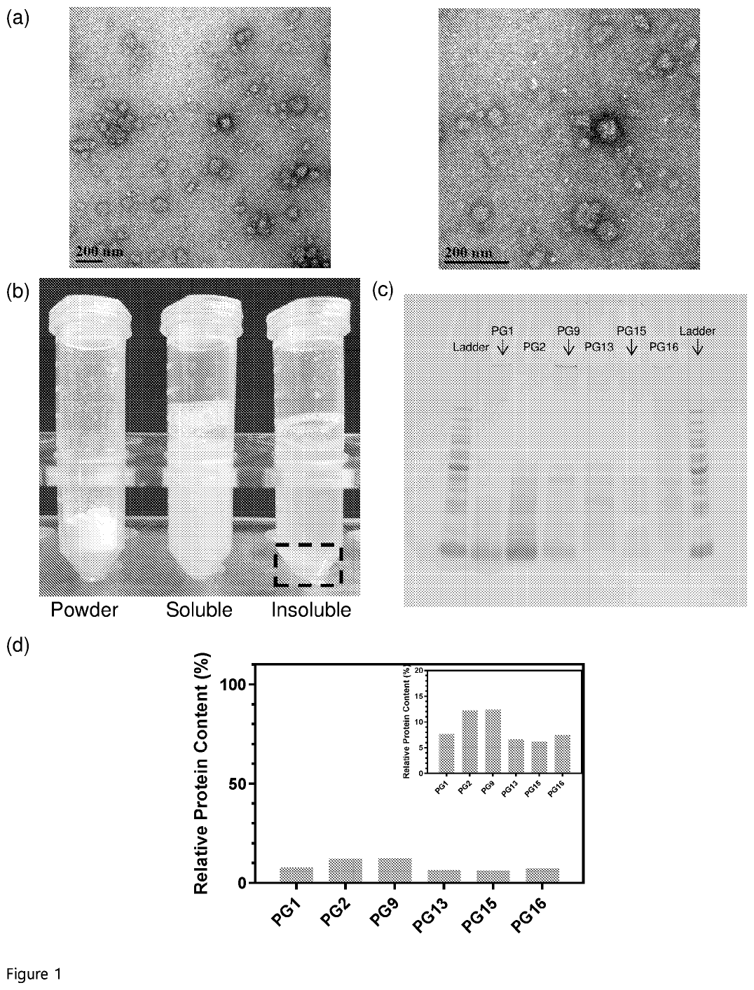

Solubility of 16 different PG extracts (PG1 to 16) were tested. PG1, 2, 5, 8, 9, 11, 13, 15, and 16 were soluble up to 20 mg/mL in neutral-buffered saline, a common protein biotherapeutic vehicle. FIG. 1 provides the results of these studies. Only soluble PG were used for further experiments.

### Toxicity Test

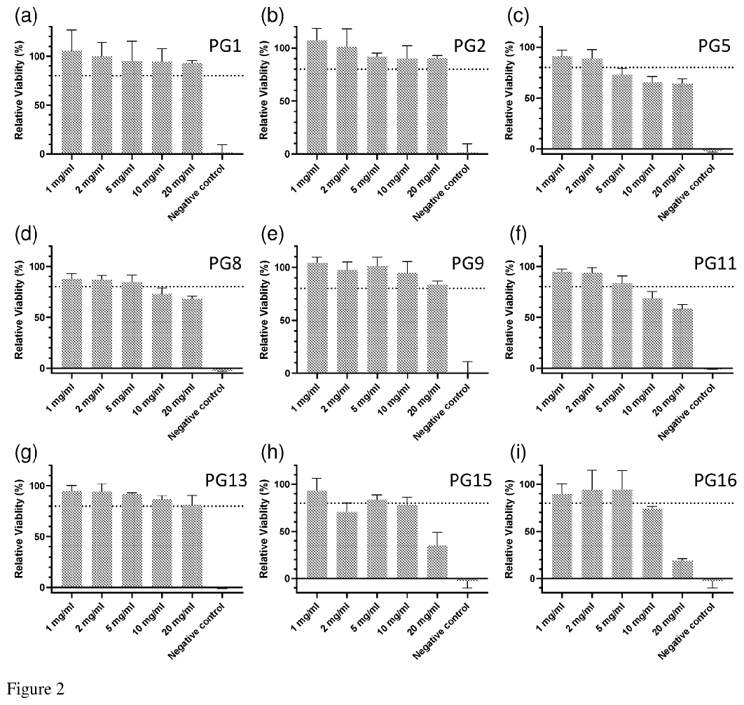

Cytocompatibility of soluble PG extracts was tested in vitro using a standard NIH 3T3 metabolic activity assay. More than 80% of NIH3T3 fibroblast cells were viable after 24 h of culture in media supplemented with or without PG1, 2, 9, and 13, which indicates that these PGs were cytocompatible. In contrast, PG5, 8, and 11 exhibited mild cytotoxicity at the highest concentrations tested, while PG15 and 16 demonstrated more cytotoxicity. FIG. 2 provides the graph showing the cytotoxicity results of the samples tested.

### Protein Contaminant Test

The different PG extracts had different amounts of protein contaminants. In particular, based on qualitative SDS-PAGE analyses, PG1, 9, and 16 had higher molecular weight (MW) protein contaminants when compared to the other PG extracts. PG2 had stronger band intensity compared to the other extracts, suggesting it had the most protein contaminants, while PG13 and 15 had weaker band intensity than the other extracts. The quantitative 660 nm assay generally aligned with the qualitative SDS-PAGE analysis. In particular, the 660 nm assay determined that PG1, 2, 9, 13, 15, and 16 contained 7.75%, 12.25%, 12.44%, 6.58%, 6.21%, and 7.51% protein content, respectively. FIGS. 1C and 1D provide the results of the contaminant test.

### Preservation Test (Fixed PG Concentration)

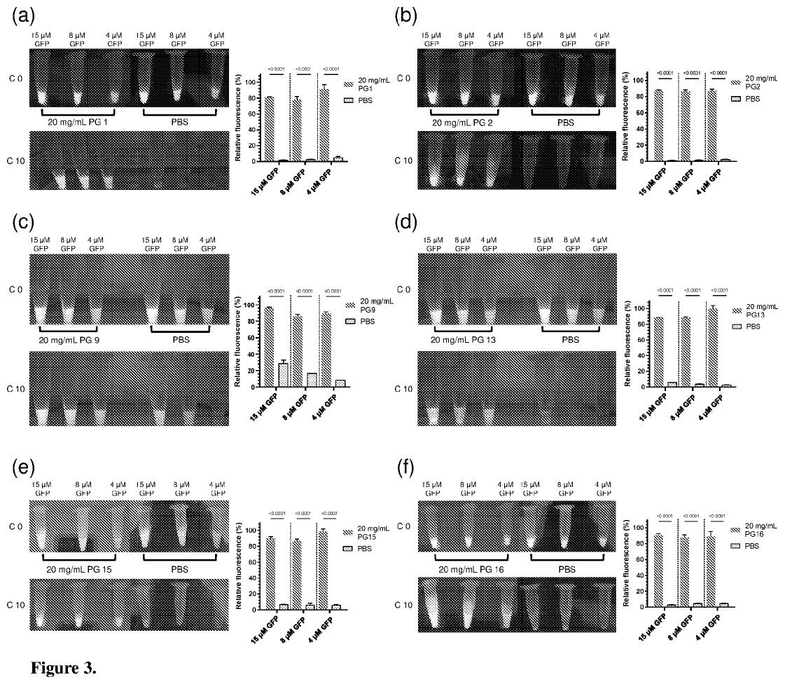

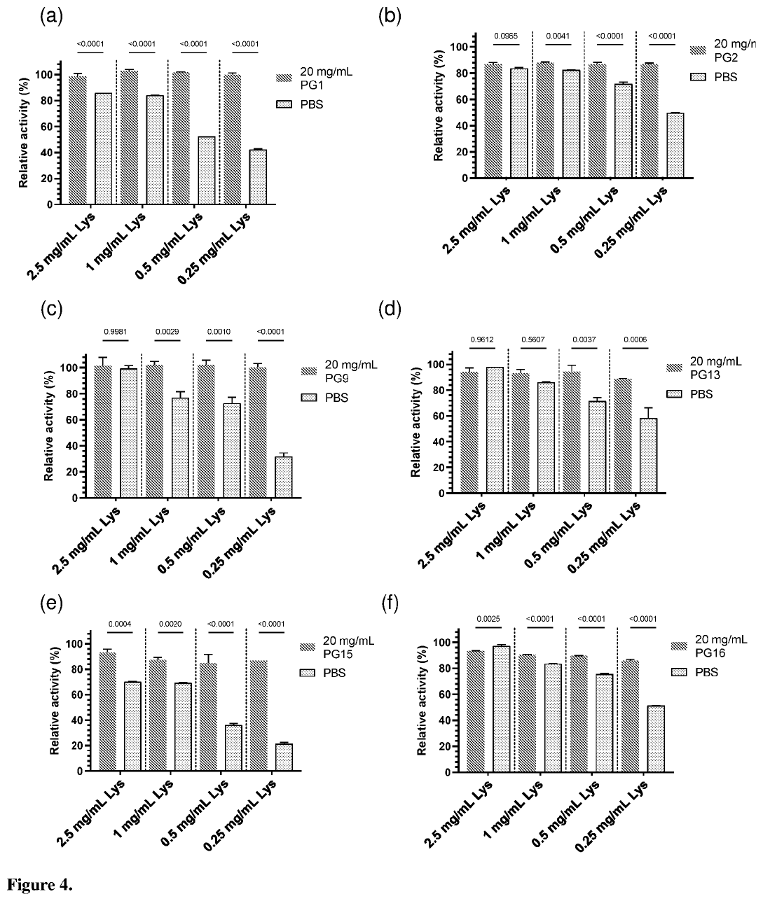

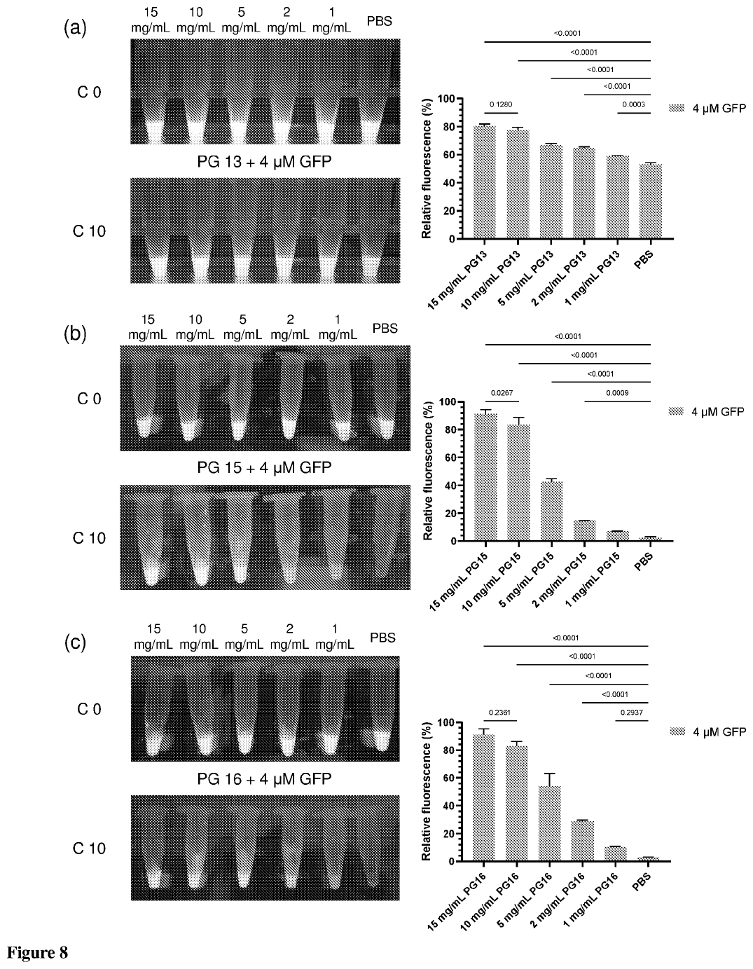

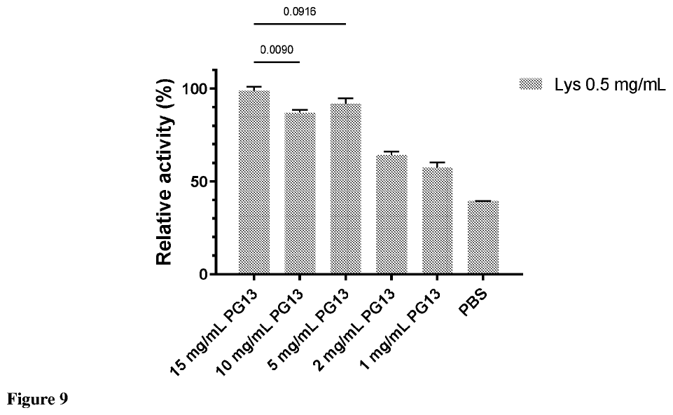

Adding PG extracts to the protein solutions stabilized protein activity during lyophilization. To assess the stabilizing ability of the different PG extracts, maintenance of GFP and lysozyme activity was evaluated first. 20 mg/mL PG maintained at least 80% of GFP activity after 10 lyophilization cycles, which was significantly greater than GFP activity in solutions without PG (see FIG. 3, supplementary data of GFP preservation is presented in FIG. 8). PG13 was the most effective stabilizer for GFP, having maintained 99% of the fluorescence activity after 10 lyophilization cycles. Likewise, lysozyome activity in solutions with 20 mg/mL PG was significantly greater than that in solutions without PG after 10 lyophilization cycles, with PG1 demonstrating the most potent protein-stabilizing activity (see FIG. 4, supplementary data of lysozyme preservation is shown in FIG. 9). For both GFP and lysozyme, the minimum PG concentration needed to stabilize activity was ˜10 mg/mL.

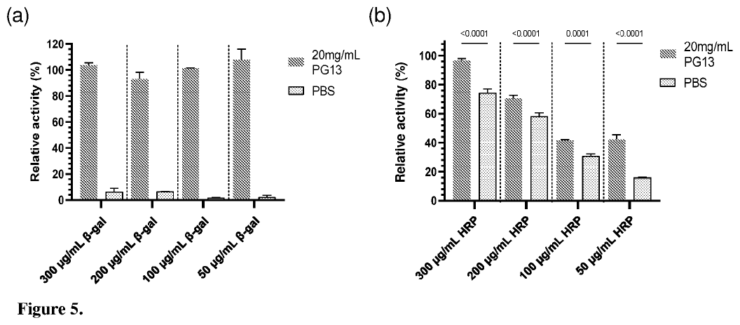

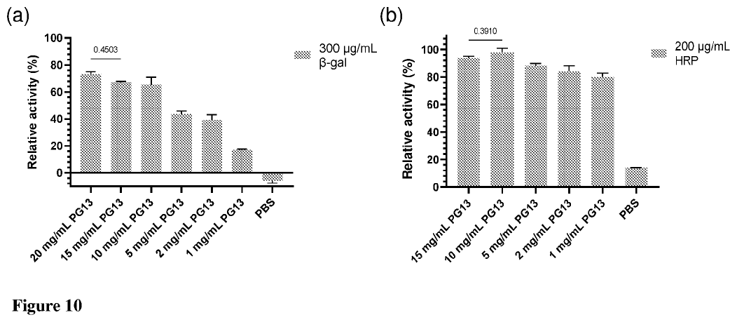

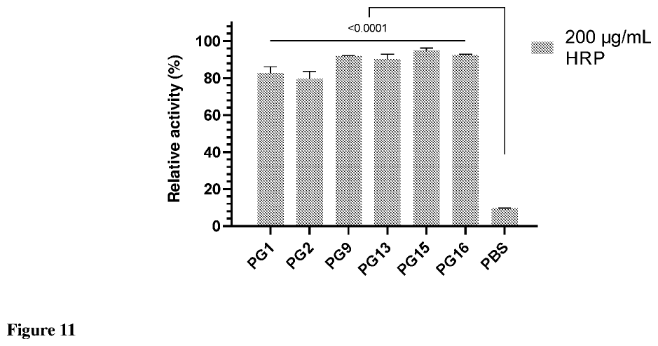

Based on these data, the effectiveness of PG13 to stabilize HRP and b-Gal activity was tested. 20 mg/mL PG13 maintained nearly 100% of b-Gal activity in solutions with 50-300 μg/mL of protein after 4 lyophilization cycles, which was significantly greater than b-Gal activity in samples without PG. Although HRP was more sensitive to lyophilization than GFP, lysozyme, and b-Gal, including 20 mg/mL PG13 in lyophilized samples with 50-300 μg/mL HRP demonstrated greater activity that that of samples without PG. See results provided in FIG. 5 and supplementary data provided in FIGS. 10 and 11.

Comparison with Common Excipients

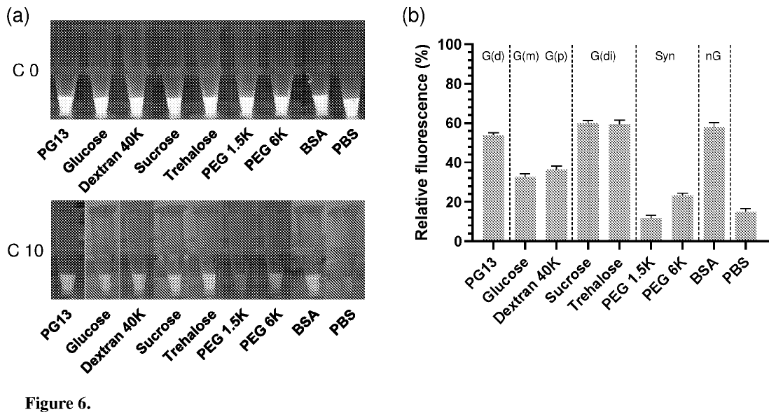

As is shown in FIG. 6, PG was an equivalent or better protein stabilizer than other common excipients. In particular, GFP solutions with added PG13 demonstrated comparable fluorescence activity after 10 cycles of lyophilization as solutions with added sucrose and trehalose, which are glucoside dimers. PG13, sucrose, and trehalose were also comparable to BSA with regard to GFP stabilizing activity. Notably, GFP solutions with added PG13 demonstrated significantly greater fluorescence activity than solutions with added PEG, which was not an effective GFP stabilizer, as well as solutions with added glucose or dextran, where the latter is a glucose polymer. These data suggested that the dendrimer architecture of glucose in PG is important for the protein stabilizing effect.

### Structural Analysis (FT-IR)

The FT-IR was used to assess the structure of lysozyme after 10 cycles of lyophilization. The A1650 cm⁻¹ and A1550 cm⁻¹ bands correspond to amide1 and amide2, respectively. The amide1 band represents the folded signal of the protein, while the amide2 band represents the backbone signal of the protein. When the protein loses its structure, the amide1 folded signal is more likely to lose its intensity. By comparing the shape and ratio of the amide1 and amide2 bands, the protein structure can be analyzed.

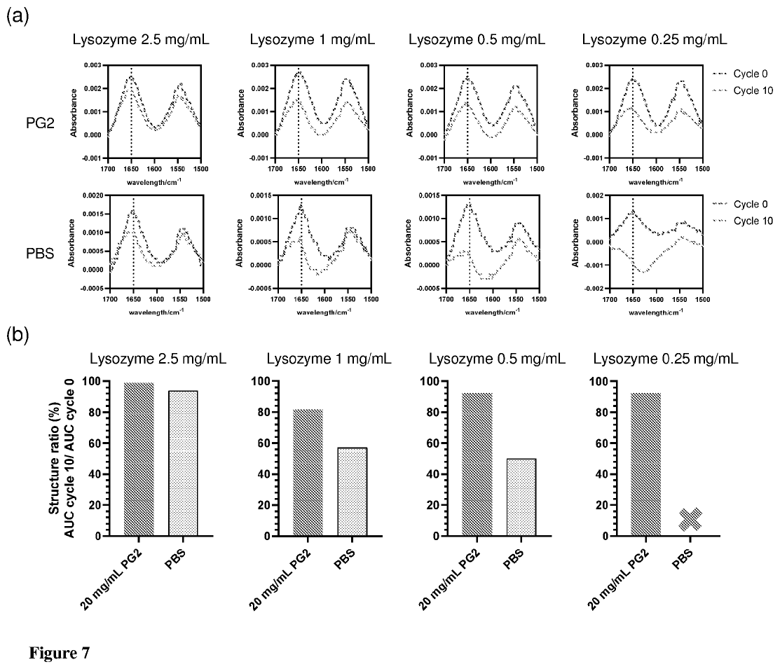

As shown in FIG. 7, the results reveal that in the PG2 group, the shape of the graph remained stable and the ratio between the amide1 and amide2 bands was maintained. However, in the PBS group, the shape of the graph collapsed, especially at 0.25 mg/ml of lysozyme, and the ratio of amide1 band decreased at all concentrations after cycle 10. Therefore, the findings suggest that the lysozyme structure was preserved with PG2, whereas the PBS group lost its structure after 10 cycles of lyophilization. See FIG. 7A.

Quantitative analysis was performed by calculating the area under the absorbance curve (AUC), so preserved lysozyme structure was compared (see FIG. 7B. The data suggesting that PG2 excipients could preserve up to 92.3% of protein structural integrity at 0.5 mg/ml of lysozyme, in contrast to 50% in control groups without PG. The structural integrity of PG2 at 0.25 mg/mL of lysozyme was 92%. However, it was not possible to compare it with the PBS group as the shape of the graph had collapsed, and thus, the AUC was unavailable. The FT-IR measurements indicated that PG stabilizes the protein by preserving its structure, which is typically susceptible to disrupted during the lyophilization process.

### EQUIVALENTS

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification and the claims are to be understood as being modified in all instances by the term “about.” Accordingly, unless indicated to the contrary, the numerical parameters set forth herein are approximations that can vary depending upon the desired properties sought to be obtained by the present invention.

What is claimed is:

1. A formulation comprising a protein active ingredient and phytoglycogen.

2. The formulation of claim 1, wherein the formulation contains between about 1 μg/ml and about 20 mg/ml of protein active ingredient, or between about 50 μg/mL to about 300 μg/mL protein active ingredient, or about 500 mg/mL to about 3 mg/mL protein active ingredient.

3. The formulation of claim 1, wherein the phytoglycogen is corn phytoglycogen.

4. The formulation of claim 3, wherein the corn phytoglycogen is obtained from a maize mutant comprising a mutation on sugary1 (su1) gene, sugary2 (su2) and/or isoamylase2 (isa2) gene.

5. The formulation of any of claims 1-4, wherein the protein active ingredient is a therapeutic protein.

6. The formulation of claim 5, wherein the protein active ingredient is selected from the group consisting of an antibody, an antibody-drug conjugate, an enzyme, a cytokine, a neurotoxin, a fusion protein, an immunogenic protein, a PEGylated protein, and an antibody fragment.

7. The formulation of any of claims 1-4, wherein the protein active ingredient is a non-therapeutic protein.

8. The formulation of claim 6, wherein the protein active ingredient is fibrous protein, structural protein, or enzyme.

9. The formulation of any of claims 1-8, wherein the formulation contains about 0.1 mg/mL to about 30 mg/ml of phytoglycogen, or contains about 5 mg/ml to about 20 mg/mL phytoglycogen, or about 10 mg/ml to about 20 mg/mL phytoglycogen.

10. The formulation of any of claims 1-9, wherein the formulation further comprises an adjunct stabilizing excipient selected from the group consisting of polypropylene glycol, adducts of polypropylene glycol, and random copolymers comprising propylene oxide units.

11. The formulation of any of claims 1-9, wherein the formulation further comprises hydrophobically modified cellulosic polymer.

12. The formulation of claim 11, wherein the hydrophobically modified cellulosic polymer is selected from the group consisting of a methylcellulose, a hydroxypropyl methylcellulose, a hydroxypropyl cellulose, and a hydroxyethyl cellulose.

13. The formulation of any of claims 1-9, wherein the formulation further comprises a polyvinyl alcohol.

14. The formulation of claim 13, wherein the polyvinyl alcohol has a molecular weight between about 500 and 500,000 Daltons.

15. The formulation of any of claims 1-9, wherein the formulation further comprises a polyoxazoline.

16. The formulation of claim 15, wherein the polyoxazoline is selected from the group consisting of poly(2-methyl-2-oxazoline), poly(2-ethyl-2-oxazoline) and poly(2-propyl-2-oxazoline).

18. The formulation of any of claims 1-9, wherein the formulation further comprises polyvinylpyrrolidone.

19. The formulation of claim 1, wherein the formulation excludes conventional surfactants.

20. The formulation of claim 1, wherein the formulation further comprises between about 1 and 5000 ppm of a conventional surfactant.

21. The formulation of any of claims 1-20, further comprising an additional agent selected from the group consisting of preservatives, sugars, polysaccharides, arginine, proline, hyaluronidase, stabilizers, and buffers.

22. A method of improving stability in a formulation comprising a protein active ingredient by including a stability-improving amount of phytoglycogen in the formulation.

23. The method of claim 22, wherein the phytoglycogen reduces degradation of the protein active ingredient by at least 10%, as compared to a control formulation lacking the phytoglycogen.

24. The method of claim 22 or 23, wherein the stability-improving amount of phytoglycogen comprises about 0.1 mg/mL to about 30 mg/ml of phytoglycogen, or about 5 mg/mL to about 20 mg/mL phytoglycogen, or about 10 mg/mL to about 20 mg/mL phytoglycogen, or about 10 mg/mL phytoglycogen
